(19) United States
(12) Patent Application Publication
(10) Pub. No.: US 2025/0124816 A1
(43) Pub. Date: Apr. 17, 2025

(54) SIMULATING ELECTROMAGNETIC PROPERTIES OF AN ANIMATE HUMAN HEAD

(71) Applicant: COOK CHILDREN'S HEALTH CARE SYSTEM, Fort Worth, TX (US)

(72) Inventor: Christos Papadelis, Fort Worth, TX (US)

(21) Appl. No.: 18/912,943

(22) Filed: Oct. 11, 2024

Related U.S. Application Data
(60) Provisional application No. 63/590,606, filed on Oct. 16, 2023.
(60) Provisional application No. 63/604,677, filed on Nov. 30, 2023.

(51) Int. Cl. G09B 23/28; A61B 5/245; A61B 5/386

(52) U.S. Cl. CPC G09B 23/286; A61B 5/245; A61B 5/386

(57) ABSTRACT

Systems and methods according to which a plurality of dipoles embedded within a simulated human brain of a simulated human head are stimulated with electricity. In one or more embodiments, the electricity with which the plurality of dipoles are stimulated is based on a recording of an animate human head. In one or more embodiments, stimulating the plurality of dipoles with the electricity causes the simulated human head to generate one or more electromagnetic properties that simulate same of the animate human head. In one or more embodiments, the one or more electromagnetic properties are detected from the simulated human head via: a first non-invasive technique; a second non-invasive technique that is different from the first non-invasive technique; or both the first non-invasive technique and the second non-invasive technique. For example, the one or more electromagnetic properties may be detected via both the first and second non-invasive techniques simultaneously.

### CROSS-REFERENCE TO RELATED APPLICATIONS

This application claims the benefit of the filing date of, and priority to, U.S. Patent Application No. 63/590,606 (the “'606 Application”), filed Oct. 16, 2023, the entire disclosure of which is hereby incorporated herein by reference.

This application claims the benefit of the filing date of, and priority to, U.S. Patent Application No. 63/604,677 (the “'677 Application”), filed Nov. 30, 2023, the entire disclosure of which is hereby incorporated herein by reference.

### GOVERNMENT SUPPORT CLAUSE

This invention was made with government support under R01 NS104116 awarded by the National Institutes of Health. The government has certain rights in the invention.

### BACKGROUND

The present disclosure relates generally to medical simulators, and, more particularly, to apparatuses, methods, and systems for simulating the electromagnetic properties of an animate human head.

### BRIEF DESCRIPTION OF THE DRAWINGS

FIG. 1(a) diagrammatically illustrates a system including a phantom for simulating the electromagnetic properties of an animate human head, according to one or more embodiments of the present disclosure.

FIG. 1(b) illustrates a plurality of testing molds for producing conductive materials for the phantom of FIG. 1(a), which conductive materials mimic the typical (e.g., average) conductivities of the human brain, skull, and scalp, according to one or more embodiments of the present disclosure.

FIG. 1(c) is an enlarged view of one of the plurality of test molds of FIG. 1(b), according to one or more embodiments of the present disclosure.

FIG. 2 diagrammatically illustrates measured resistance values for various Si-Ca mixture ratios, according to one or more embodiments of the present disclosure.

FIG. 3(a) illustrates a brain layer extracted from co-registered CT and MRI scans of a patient, according to one or more embodiments of the present disclosure.

FIG. 3(b) illustrates a skull layer extracted from co-registered CT and MRI scans of a patient, according to one or more embodiments of the present disclosure.

FIG. 3(c) illustrates a scalp layer extracted from co-registered CT and MRI scans of a patient, according to one or more embodiments of the present disclosure.

FIG. 4(a) illustrates a simplified version of the skull layer of FIG. 3(b), according to one or more embodiments of the present disclosure.

FIG. 4(b) illustrates the simplified version of the skull layer of FIG. 4(a) from a different viewing angle, according to one or more embodiments of the present disclosure.

FIG. 5(a) diagrammatically illustrates a phantom including a base, a brain layer, a skull layer, and a scalp layer, together with a brain mold, a skull mold, and a scalp mold for forming the brain layer, the skull layer, and the scalp layer, respectively, according to one or more embodiments of the present disclosure.

FIG. 5(b) diagrammatically illustrates the base and the brain layer of FIG. 5(a), according to one or more embodiments of the present disclosure.

FIG. 6(a) illustrates a first stage in the fabrication of the phantom of FIG. 5(a), which first stage includes molding the brain layer to the base, according to one or more embodiments of the present disclosure.

FIG. 6(b) illustrates a second stage in the fabrication of the phantom of FIG. 5(a), which second stage includes molding the skull layer to the brain layer, which brain layer is molded to the base, according to one or more embodiments of the present disclosure.

FIG. 6(c) illustrates a third stage in the fabrication of the phantom of FIG. 5(a), which third stage includes molding the scalp layer to the skull layer, which skull layer is molded to the brain layer, and which brain layer is molded to the base, according to one or more embodiments of the present disclosure.

FIG. 7(a) illustrates a 3D computer model for accurately positioning a plurality of dipoles within the base of FIGS. 6(a)-(c), according to one or more embodiments of the present disclosure.

FIG. 7(b) illustrates the fabricated base of FIG. 7(a) with the plurality of dipoles positioned therethrough, according to one or more embodiments of the present disclosure.

FIG. 7(c) illustrates the base and the dipoles of FIG. 7(b) assembled into the fabricated brain mold, according to one or more embodiments of the present disclosure.

FIG. 8 illustrates the fabricated base, brain mold, skull mold, and scalp mold of FIG. 5(a), according to one or more embodiments of the present disclosure.

FIG. 9(a) illustrates the skull mold of FIG. 8, according to one or more embodiments of the present disclosure.

FIG. 9(b) illustrates the scalp mold of FIG. 8, according to one or more embodiments of the present disclosure.

FIG. 10(a) illustrates the fabricated base and brain layer of FIG. 5(a), according to one or more embodiments of the present disclosure.

FIG. 10(b) illustrates the fabricated base, brain layer, and skull layer of FIG. 5(a), according to one or more embodiments of the present disclosure.

FIG. 10(c) illustrates the fabricated base, brain layer, skull layer, and scalp layer of FIG. 5(a), according to one or more embodiments of the present disclosure.

FIG. 11(a) illustrates a junction box to which the dipoles in the phantom are wired, according to one or more embodiments of the present disclosure.

FIG. 11(b) illustrates a pedestal for supporting the junction box and phantom of FIG. 11(a), according to one or more embodiments of the present disclosure.

FIG. 12(a) illustrates the fabricated base assembled with a plurality of guide tubes, according to one or more embodiments of the present disclosure.

FIG. 12(b) illustrates the fabricated base and guide tubes of FIG. 12(a) from a different viewing angle, according to one or more embodiments of the present disclosure.

FIG. 12(c) illustrates the fabricates base of FIGS. 12(a)-(b) with a removable part that provides clearance for insertion of the dipoles, according to one or more embodiments of the present disclosure.

FIG. 13 illustrates a dipole for insertion through one of the guide tubes of FIGS. 12(a)-(b), according to one or more embodiments of the present disclosure.

FIG. 14(a)(1) illustrates a plurality of dipoles implanted into the brain layer, according to one or more embodiments of the present disclosure.

FIG. 14(a)(2) illustrates a first non-invasive system, namely a magnetoencephalography (“MEG”) system, which records the phantom when the implanted dipoles of FIG. 14(a)(1) are stimulated with the signal of FIG. 14(b), according to one or more embodiments of the present disclosure.

FIG. 14(a)(3) illustrates a second non-invasive system, namely a high-definition electroencephalography (“HD-EEG”) system, which records the phantom when the implanted dipoles of FIG. 14(a)(1) are stimulated with the signal of FIG. 14(b), according to one or more embodiments of the present disclosure.

FIG. 14(b) diagrammatically illustrates a signal used to stimulate the implanted dipoles of FIG. 14(a)(1), the signal including interictal spike epochs taken from a medically refractory epilepsy patient, according to one or more embodiments of the present disclosure.

FIG. 15(a) diagrammatically illustrates a recording taken by the MEG system of FIG. 14(a)(2), according to one or more embodiments of the present disclosure.

FIG. 15(b) diagrammatically illustrates a recording taken by the HD-EEG system of FIG. 14(a)(3), according to one or more embodiments of the present disclosure.

FIG. 15(c) diagrammatically illustrates medically imaged locations of the implanted dipoles, together with a source localization based on the recording of FIG. 15(a) and a source localization based on the recording of FIG. 15(b), according to one or more embodiments of the present disclosure.

FIG. 16(a) illustrates a tangential dipolar source, according to one or more embodiments of the present disclosure.

FIG. 16(b) illustrates a radial dipolar source, according to one or more embodiments of the present disclosure.

FIG. 17(a) illustrates another signal used to stimulate the implanted dipoles of FIG. 14(a)(1), the another signal including epilepsy biomarkers taken from a fast seizure onset in an epileptic patient, according to one or more embodiments of the present disclosure.

FIG. 17(b) illustrates another signal used to stimulate the implanted dipoles of FIG. 14(a)(1), the another signal including epilepsy biomarkers taken from a slow seizure onset in an epileptic patient, according to one or more embodiments of the present disclosure.

FIG. 18 is a flow diagram of a method for implementing one or more embodiments of the present disclosure.

FIG. 19 diagrammatically illustrates a computing node for implementing one or more embodiments of the present disclosure.

### DETAILED DESCRIPTION

The present disclosure introduces a physical head model (or “phantom”) usable for training electrophysiologists, medical and PHD students, as well as electroencephalography (“EEG”) and magnetoencephalography (“MEG”) technologists and technicians, to educate such individuals on electric and magnetic source imaging aspects. The phantom (also referred to herein as a simulated human head) resembles a human head, containing three layers, namely a simulated brain (also referred to herein as a brain layer), a simulated skull (also referred to herein as a skull layer), and a simulated scalp (also referred to herein as a scalp layer). The geometry and electrical properties of each of the three layers of the phantom are similar to the corresponding geometry/properties of real human tissue. The simulated brain layer includes a plurality of dipolar sources (e.g., 10) (also referred to herein as dipoles) in each hemisphere, which dipolar sources are implanted at locations representing different deep and superficial lobes of the brain. Each dipolar source is capable of being stimulated individually with electrical signal(s), which may be, include, or be based on brain signal(s) that have been recorded intracranially from patient(s). In this manner, the phantom is capable of simulating the electromagnetic properties of an animate human head by generating electrophysiological signals having similar characteristics with the actual signals generated by an animate human brain.

As an example, in some instances, the phantom generates electrophysiological signals having similar characteristics to the resting-state human brain activity of a healthy and typically-developing child or adult human patient. As another example, in other instances, the phantom generates electrophysiological signals having similar characteristics to the interictal and ictal activity of an epileptic child or adult human patient. Non-invasive functional brain imaging data, such as MEG and EEG, is recordable from the phantom to thereby assess the quality of such recording(s). Specifically, the phantom facilitates reliable assessment of the localization abilities of non-invasive technologies, such as MEG and EEG, in different clinical and research scenarios, and also provides a tool for academic, commercial, and educational purposes.

There is a growing body of in-vivo studies for developing and improving methods that localize the source (foci) of brain electrical activity using functional brain imaging systems, such as high-definition EEG (“HD-EEG”) and MEG. However, these studies lack a solid ground truth for the exact location of the brain electrical activity. The phantom of the present disclosure fills this gap by enabling the reproduction of the brain electrical activity in predefined locations within a realistically-fabricated head model, thereby serving as a ground truth for the evaluation of brain activity source localization methods. Another area, which is evolving rigorously, is the development of artificial intelligence (“AI”) and machine learning (“ML”) algorithms for detection of biomarkers for different neurological disorders, such as epilepsy, from brain recordings. These biomarkers come with different shapes and electrical characteristics, making them cumbersome to detect. Since the phantom of the present disclosure is capable of accurately reproducing electrophysiological signals as desired, it can also be used as a ground truth tool for performance assessment of biomarker detection algorithms. Finally, the phantom facilitates quantitative assessment of the recording quality of functional brain modalities such as HD-EEG, MEG, or other non-invasive brain activity recording devices/systems.

The quality of such recordings is highly affected by ambient noise, whether the brain activity originates from a deep or superficial brain source, and the orientation of the neurons that fire the electrical signals. The phantom is capable of reproducing electrical brain signals from predefined deep to superficial sources. Moreover, the phantom is capable of being recorded by conventional clinical MEG and EEG systems, which enables the assessment of the quality of the recordings from deep to superficial sources.

In summary, the phantom of the present disclosure provides the medical community with commercial, academic, and educational benefits. The phantom is a realistic 3D printed head model that includes materials resembling the electrophysiological properties of a human brain, skull, and scalp layers. The phantom addresses the lack of a solid ground truth for developing, improving, and assessing brain activity source localization methods and Al biomarker detection algorithms. The phantom also serves as a pragmatic tool for evaluating the performance of functional brain imaging modalities, such as EEG and MEG, and even combined modalities such as simultaneous MEG/EEG recordings. Finally, the phantom is reproducible, meaning that realistic phantoms can be constructed based on magnetic resonance imaging (MRI) of specific patients, making the phantom beneficial for presurgical evaluations of patients.

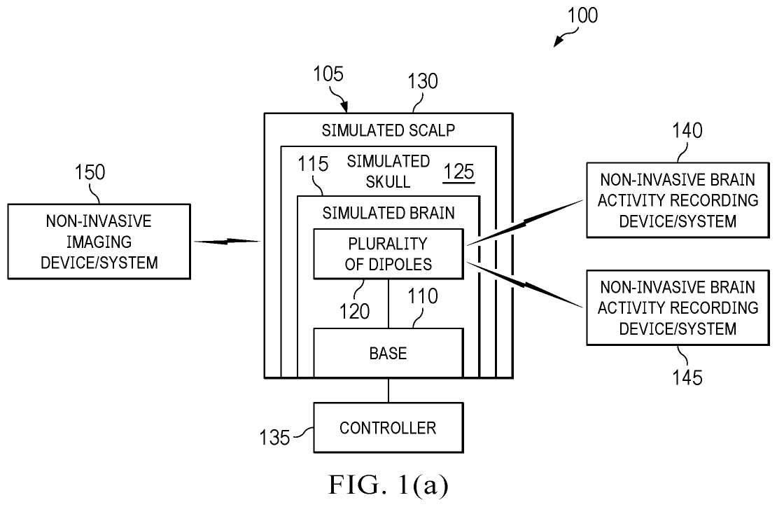

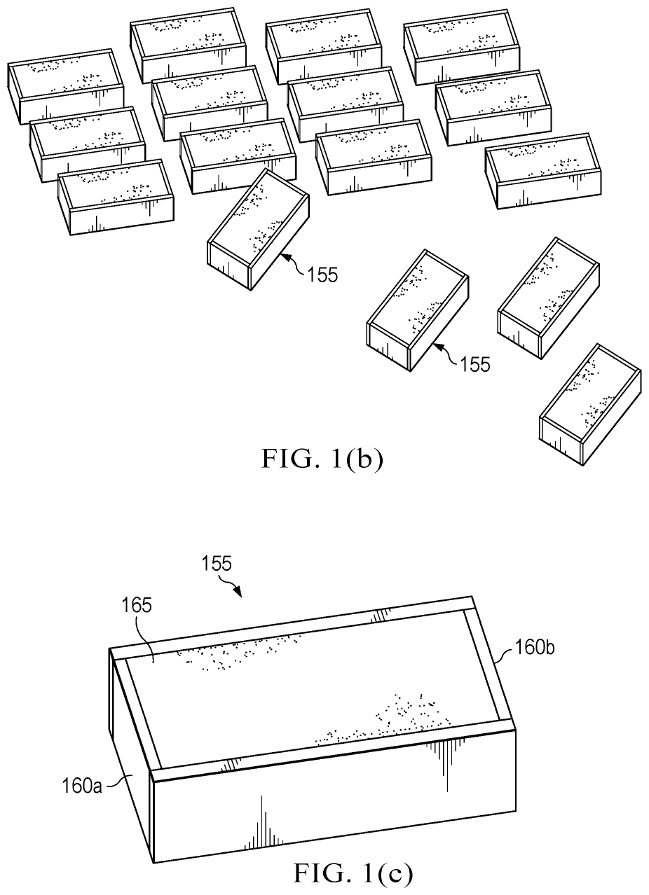

Referring to FIG. 1(a), in one or more embodiments, a system 100 includes a phantom 105, wherein the phantom 105 simulates the electromagnetic properties of an animate human head and includes a base 110, a simulated brain 115, a plurality of dipoles 120, a simulated skull 125, and a simulated scalp 130. The plurality of dipoles 120, which are each embedded through the base 110 into the simulated brain 115 (as will be described in more detail below), are connected to a controller 135. The controller 135 is adapted to communicate signal(s) to the plurality of dipoles 120, which signal(s) stimulate the plurality of dipoles 120; such signal(s) may be obtained, for example, from a non-invasive brain activity recording of an animate human head (i.e., a live human patient).

In one or more embodiments, a first non-invasive brain activity recording device/system 140 is adapted to detect one or more electromagnetic properties from the phantom 105 when the plurality of dipoles 120 are stimulated by the signal(s) communicated from the controller 135. For example, the first non-invasive brain activity recording device/system 140 may be, include, or be part of an MEG machine. For another example, additionally, or alternatively, the first non-invasive brain activity recording device/system 140 may be, include, or be part of an EEG machine. In one or more embodiments, the first non-invasive brain activity recording device/system 140 is further adapted to derive a source localization for one or more of the plurality of dipoles 120 based on the one or more electromagnetic properties detected from the phantom 105.

Additionally, or alternatively, in one or more embodiments, a second non-invasive brain activity recording device/system 145 is adapted to detect one or more electromagnetic properties from the phantom 105 when the plurality of dipoles 120 are stimulated by the signal(s) communicated from the controller 135. For example, the second non-invasive brain activity recording device/system 145 may be, include, or be part of an EEG machine. For another example, additionally, or alternatively, the second non-invasive brain activity recording device/system 145 may be, include, or be part of an MEG machine. In one or more embodiments, the second non-invasive brain activity recording device/system 145 is further adapted to derive a source localization for one or more of the plurality of dipoles 120 based on the one or more electromagnetic properties detected from the phantom 105.

A non-invasive imaging device/system 150 is adapted to determine the physical location(s) of the one or more of the plurality of dipoles 120 within the phantom 105 based on medical imaging. For example, the non-invasive imaging device/system 150 may be, include, or be part of a magnetic resonance imaging (“MRI”) machine. For another example, additionally, or alternatively, the non-invasive imaging device/system 150 may be, include, or be part of a computed tomography (“CT”) machine. In one or more embodiments, the physical location(s) of the one or more of the plurality of dipoles 120 within the phantom 105, as determined by the non-invasive imaging device/system 150 (based on medical imaging), are then used to assess an accuracy of the first non-invasive brain activity recording device/system 140, the second non-invasive brain activity recording device/system 145, or both.

The phantom 105 must be constructed from conductive materials mimicking the typical (e.g., average) conductivities of the human brain, skull, and scalp, respectively, in order to be useful for EEG and MEG recordings. For example, a mixture of silicone and chopped carbon fibers (“Si-Ca Mix”), combined in different mixing ratios, can be used to achieve the required conductivity values. The required mixing ratios are determined by testing for a range of conductivities typical of the selected target tissues.

Accordingly, turning also to FIGS. 1(b)-(c), with continuing reference to FIG. 1(a), in one or more embodiments, testing molds 155 are made (e.g., 3D printed) using a special resin. The molds 155 each have the same geometry (defining a volume of about 3×2×6 cm), including two (2) faces 160a-b (or “plates”), created using adhesive copper tape, at two (2) conductive extremities, thereby producing the same cross-section and length between the two (2) copper plates for each mold 155. The volume of each mold 155 is completely filled with Si-Ca Mix 165, with each batch of samples being structured using three (3) different samples per mixing ratio, and five (5) different mixing ratios (i.e., ˜1%, ˜1.5%, ˜2%, ˜2.5%, and ˜3% of Ca). In order to achieve a mixing ratio as close as possible to the desired ratio, each ingredient of the mix 165 is accurately measured in weight using a jeweler scale. All of the ingredients are poured into a single cup to avoid leftovers in different containers, and to ensure weighting of only those ingredients added to the final mix. Each sample is then degassed before being compressed into a pressure pot for the curation period, minimizing the presence of bubbles that might negatively influence the final conductivity measurement. The curing time of the silicone is significantly extended by the mixture, such that stable resistivity values are achieved only after about 200 hours from pouring. To avoid any dependency on the mixing technique, each batch of silicone is mixed in the exact same way in terms of time and type of movement adopted for the mixing process. Before being added to the mixture, the chopped Carbon fibers (CF) are soaked into a 99% alcohol bath, which preserves the contact conductivity resulting from the many conductivity paths created by the small fibers suspended within the volume. Without washing and then rinsing the CF into the alcohol bath, reduced (or no) conductivity is achieved.

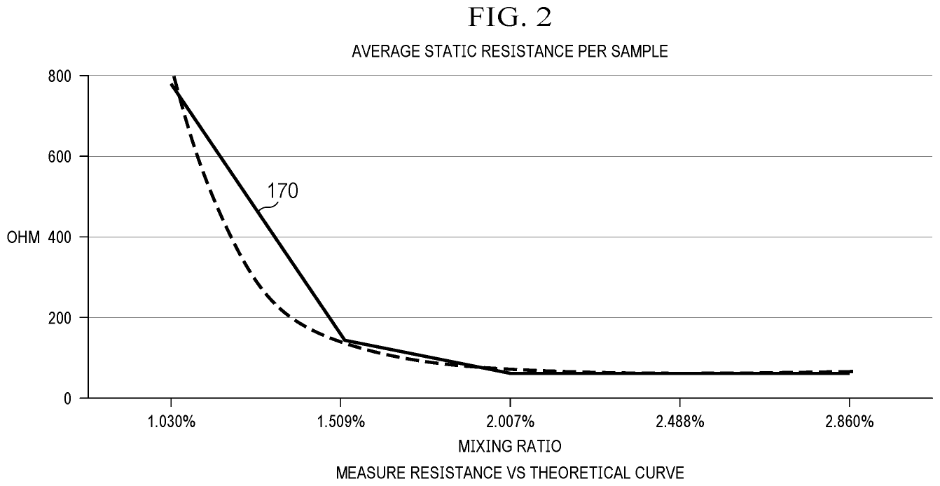

Referring to FIG. 2, with continuing reference to FIGS. 1(a)-(c), in one or more embodiments, the measured resistance values 170 illustrate that resistance saturates quickly once the mix ratio grows over 3% of Ca (i.e., CF), leaving an acceptable range around the 1% to 2% mix ratio, which coincides with the range of resistance values required for the phantom 105. Specifically, in one or more embodiments, the ideal mixing ratios are ˜1.148% for scalp and brain tissue (i.e., for the simulated scalp 130 and the simulated brain 115), and ˜1.63% for bone (i.e., for the simulated skull 125).

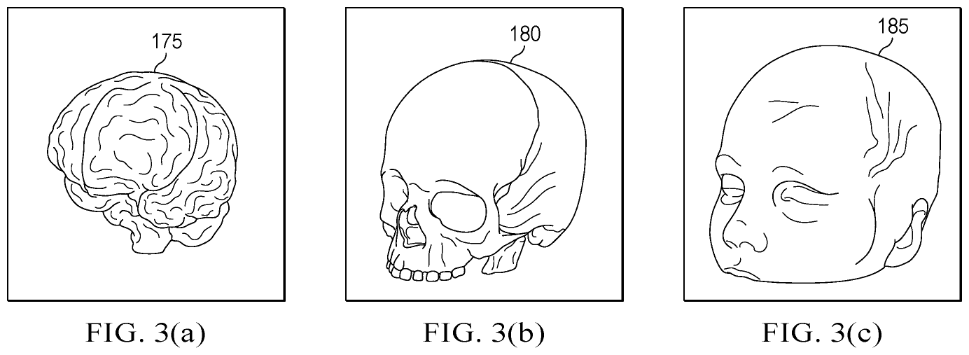

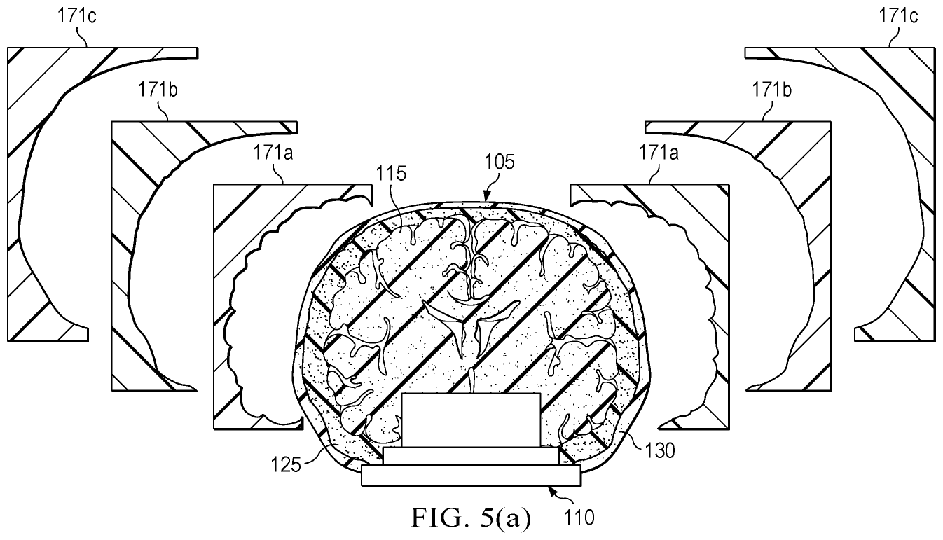

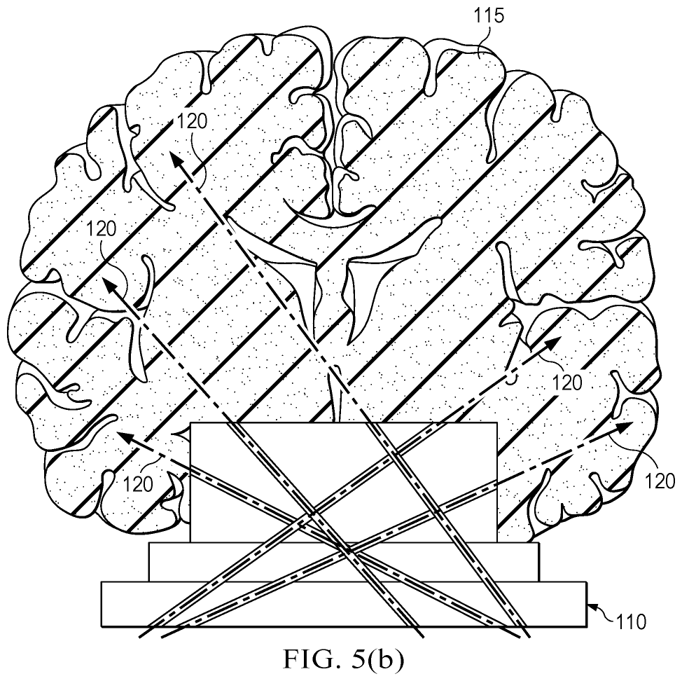

Referring to FIGS. 3(a)-(c), in one or more embodiments, molds 171a-c (shown, e.g., in FIG. 5(a)) for forming the phantom 105 itself are designed using “Slicer” to segment a patient's anatomy. Specifically, in one or more embodiments, a CT scan and an MRI scan of the patient are co-registered, and, once so co-registered, three (3) general layers are extracted, namely a brain layer 175 (i.e., volume inside the skull), a bone layer 180 (i.e., the skull), and a scalp layer 185 (i.e., outside the skull). The extracted surfaces are imported and optimized using MeshLab and Lightwave Modeler (i.e., to achieve mesh reduction, removal of duplicate vertices/faces, removal of noise/artifacts, and optimization for 3D Printing using FDA technology). The final meshes are then imported into Lightwave Modeler to facilitate creating the different molds 171a-c for forming the phantom 105. In one or more embodiments, the molds 171a-c for forming the phantom 105 are 3D printed. For example, the molds 171a-c can be 3D printed using PLA filament on a Creality CR10S 3D Printer, and using “CURA” as the slicing software.

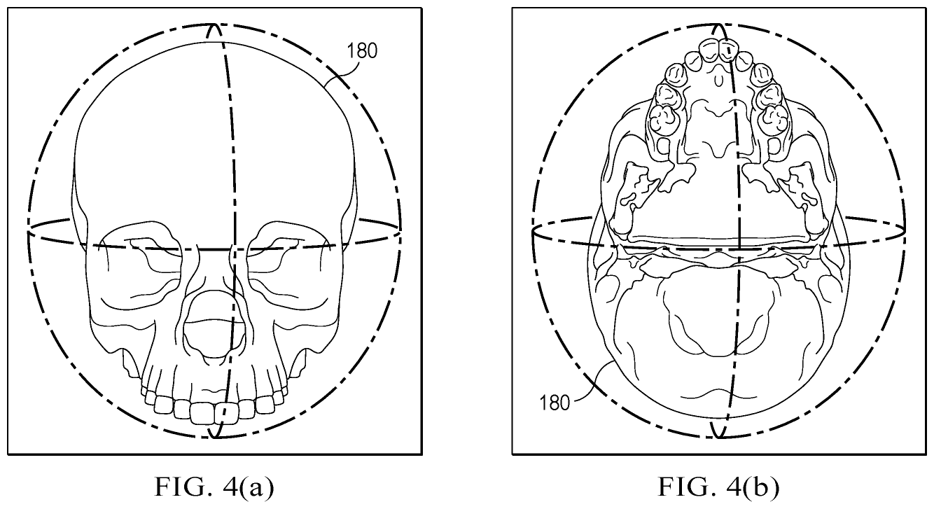

Referring to FIGS. 4(a)-(b), with continuing reference to FIG. 3(b), in one or more embodiments, the surfaces of the bone layer 180 are simplified to avoid geometries that are too complicated for the 3D Printing and casting phases to handle.

Referring to FIGS. 5(a)-(b), with continuing reference to FIGS. 3(a)-(c) and 4(a)-(b), in one or more embodiments, the molds 171a-c are designed to facilitate casting of all three (3) layers (i.e., the simulated brain 115, the simulated skull 125, and the simulated scalp 130) of the phantom 105 around the base 110, which base 110 is used to co-register the molds 171a-c across the three (3) layers, and to route the various dipoles 120 to their desired locations.

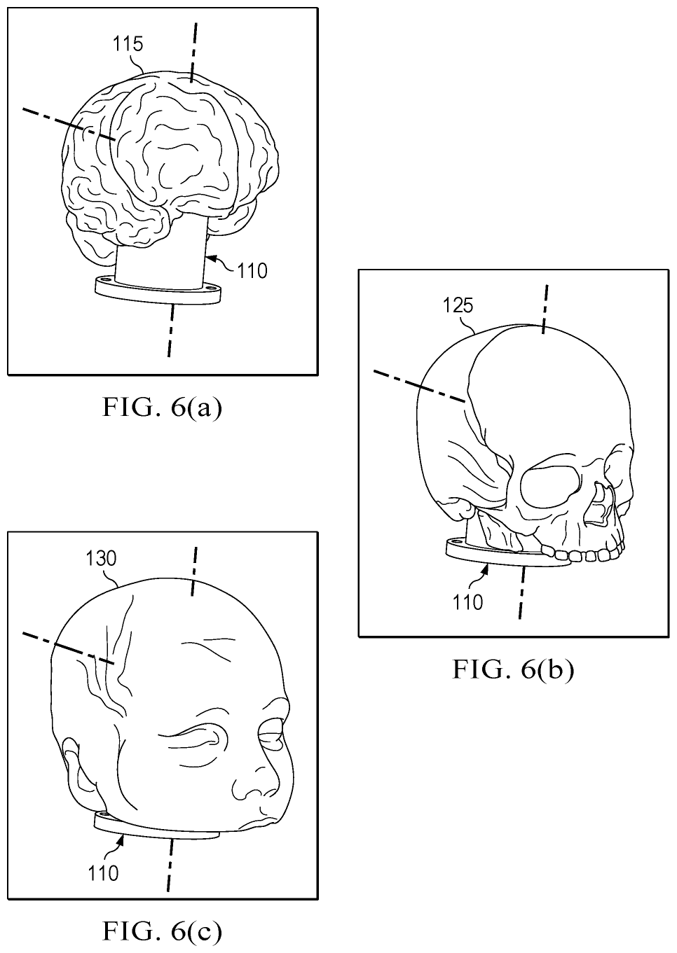

Referring to FIGS. 6(a)-(c), with continuing reference to FIGS. 5(a)-(b), in one or more embodiments, the design of the base 110 ensures accurate positioning of the dipoles 120 and of the three (3) molds 171a-c, namely the brain mold 171a, the skull mold 171b, and the scalp mold 171c. Specifically, FIG. 6(a) illustrates a first stage in the fabrication (in which the brain mold 171a has been utilized to cast the simulated brain 115 onto the base 110), FIG. 6(b) illustrates a second stage in the fabrication (in which the skull mold 171b has been utilized to cast the simulated skull 125 onto the simulated brain 115 and the base 110), and FIG. 6(c) illustrates a third stage in the fabrication (in which the scalp mold 171c has been utilized to cast the simulated scalp 130 onto the simulated skull 125, the simulated brain 115, and the base 110).

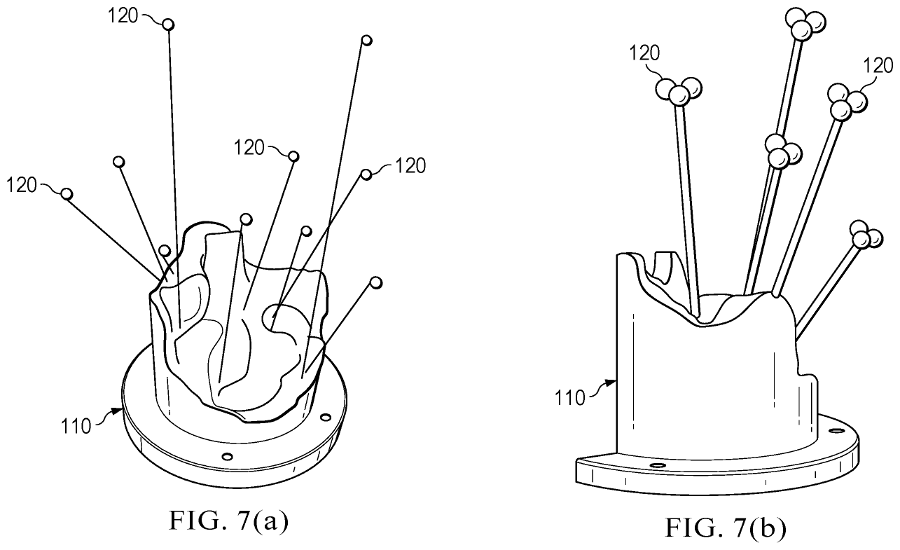

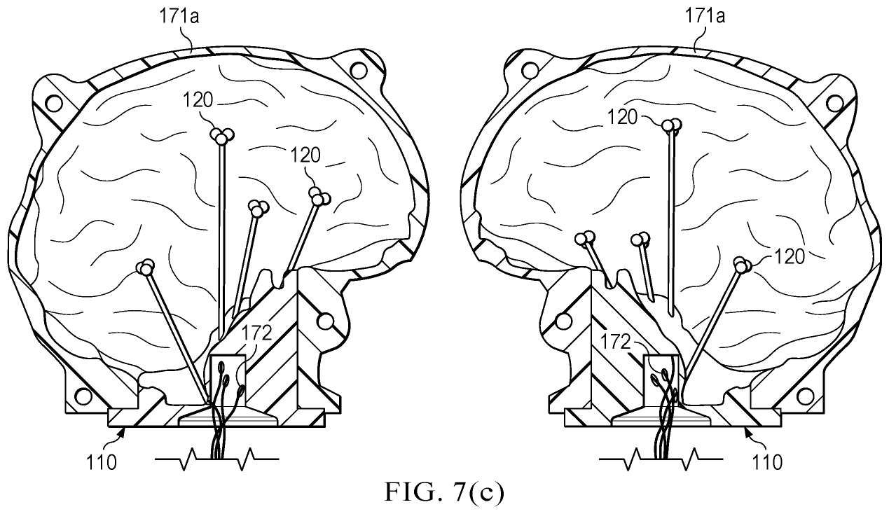

Referring to FIGS. 7(a)-(c), with continuing reference to FIGS. 5(a)-(b) and 6(a)-(c), in one or more embodiments, the base 110 of the phantom 105 serves as the centerpiece of the fabrication process, keeping the dipoles 120 and molds 171a-c in place through the different stages of fabrication. The design of the base 110 begins with the accurate positioning of the dipoles 120 within the brain layer 175 (shown in FIG. 3(a)) using Slicer, as shown in FIG. 7(a). The base 110 is then designed with the necessary holes 172 to route the dipoles 120 to their desired locations, as shown in FIGS. 7(b)-(c).

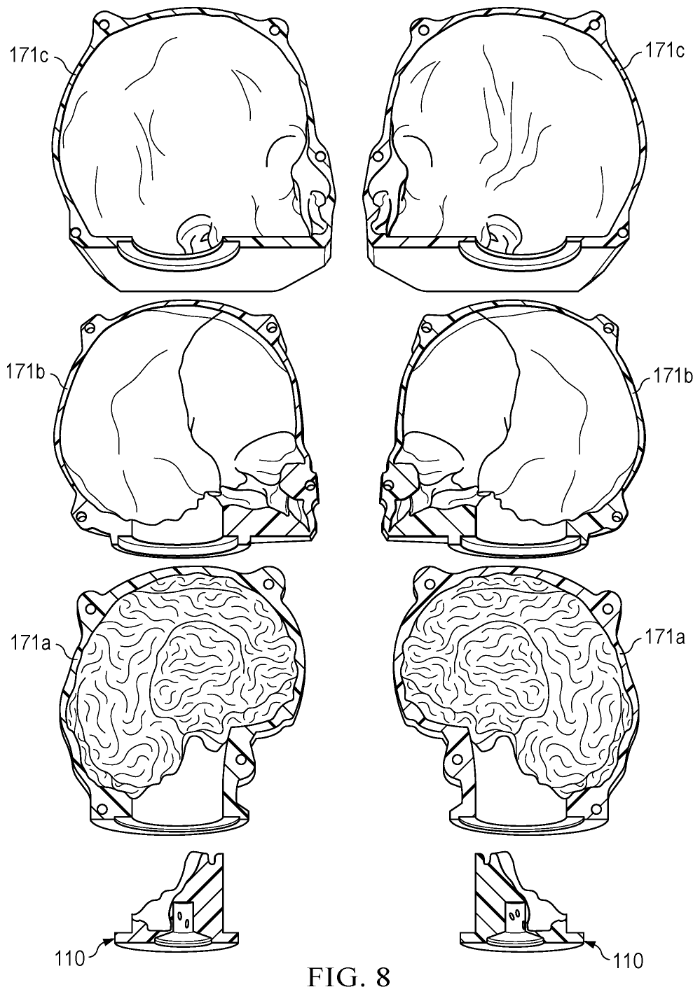

Referring to FIG. 8, with continuing reference to FIGS. 7(a)-(c), in one or more embodiments, each of the molds 171a-c is built around the base 110, subtracting at each stage the anatomical surface and then the base 110. The base 110 is designed to route all of the different dipoles 120 from a central duct to their desired locations using, for example, 3 mm carbon fibers tubes. FIG. 8 illustrates the set of all of the molds 171a-c together with the base 110.

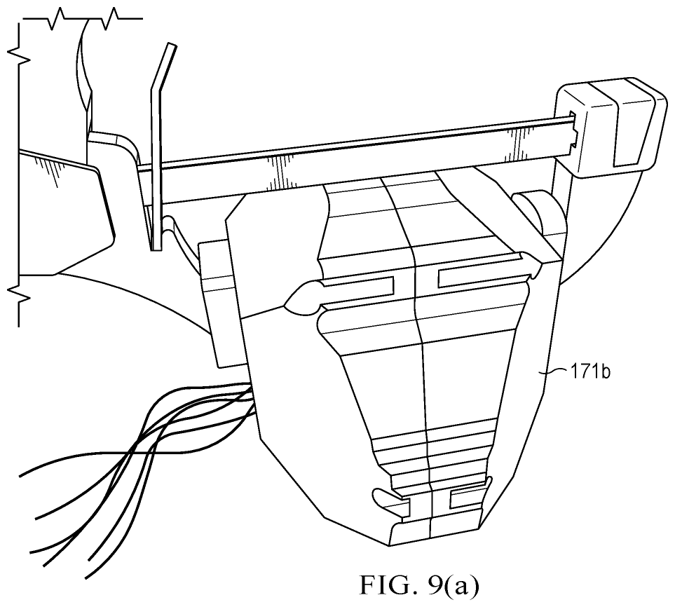

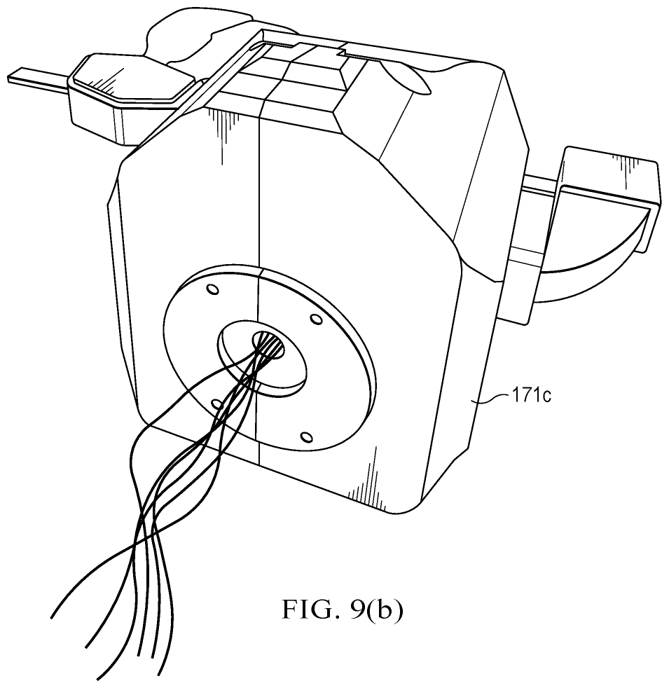

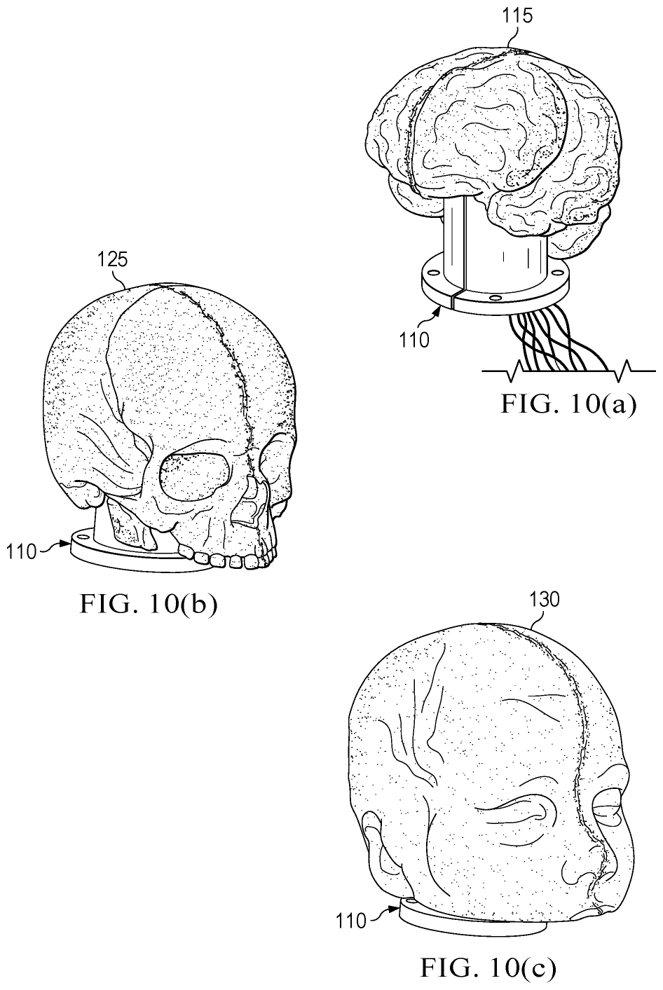

Referring to FIGS. 9(a)-(b) and 10(a)-(c), with continuing reference to FIG. 8, in one or more embodiments, at a first stage, the two halves of the base 110 are mounted into the corresponding two halves of the brain mold 171a using a bottom lip and alignment pins. The corresponding ratio (e.g., 1.148%) of Si-Ca Mix 165 is poured into the two halves of the brain mold 171a, which are joined and clamped together once the Si-Ca Mix 165 is at least partially cured (e.g., about 50%). The brain mold 171a is subsequently removed from the simulated brain layer 115 and the base 110, leaving the simulated brain layer 115 molded to the base 110, as shown in FIG. 10(a). Next, the steps are repeated for a second stage, at which the corresponding ratio (e.g., 1.63%) of Si-Ca Mix 165 is poured in the two halves of the skull mold 171b, which are joined/clamped together and aligned through the bottom lip of the base 110 and the alignment pins, as shown in FIG. 9(a). The skull mold 171b is subsequently removed from the simulated skull layer 125, leaving the simulated skull layer 125 and the simulated brain layer 115 molded to the base 110, as shown in FIG. 10(b). Finally, the steps are repeated for a third stage, at which the corresponding ratio of Si-Ca Mix 165 (e.g., 1.148%) is poured in the two halves of the scalp mold 171c, which are joined/clamped together and aligned through the bottom lip of the base 110 and the alignment pins, as shown in FIG. 9(b). The scalp mold 171c is subsequently removed from the simulated scalp layer 130, leaving the simulated scalp layer 130, the simulated skull layer 125, and the simulated brain layer 115 molded to the base 110, as shown in FIG. 10(c).

Referring still to FIGS. 10(a)-(c), with continuing reference to FIGS. 8 and 9(a)-(b), the first, second, and third stages are illustrated after removal of the corresponding molds 171a-c to reveal the simulated brain layer 115 (shown in FIG. 10(a)), the simulated skull layer 125 (shown in FIG. 10(b)), and the simulated scalp layer 130 (shown in FIG. 10(c)), respectively, according to one or more embodiments. At each stage, after demolding, the corresponding layer of the phantom 105 requires post-processing to remove molding artifacts (i.e., leakages, superficial bubbles, silicon-CF separation, etc.). To optimize conductivity between layers of the phantom 105, the CF is exposed and wet with alcohol before pouring the next layer. For example, light sandblasting with glass powder may be applied to increase conductivity between layers by exposing the CF beneath the exterior surface. Additionally, or alternatively, another technique, such as light scraping using a razor-blade, may be applied to achieve the same (or similar) result. The alcohol prevents the silicone from adhering completely to the CF fibers, leaving them available for contact with the CF fibers in the next layer.

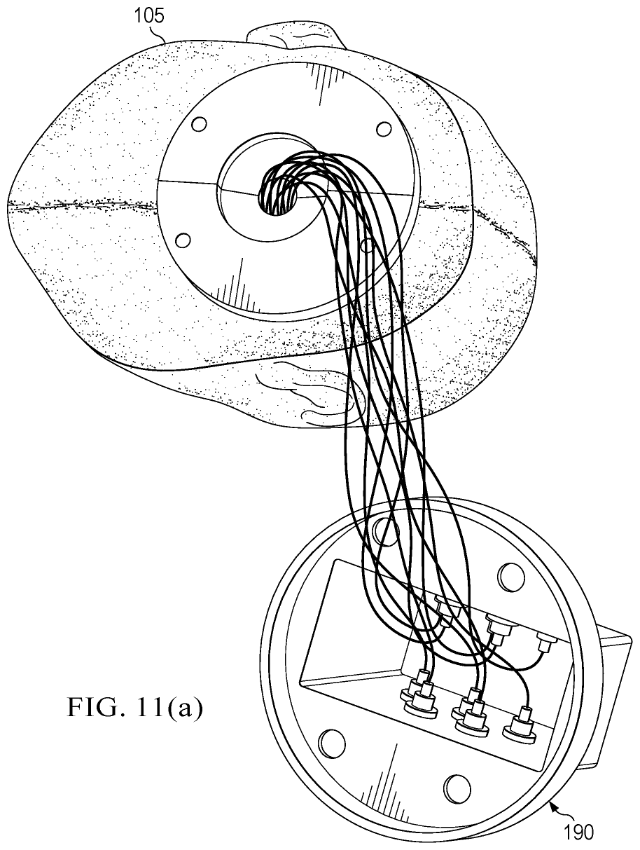

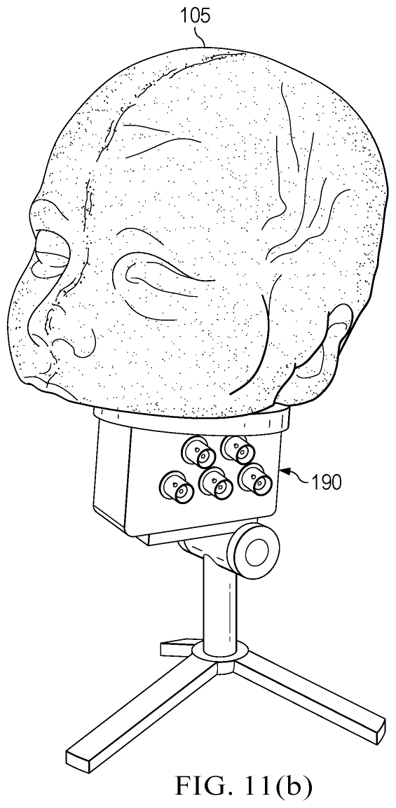

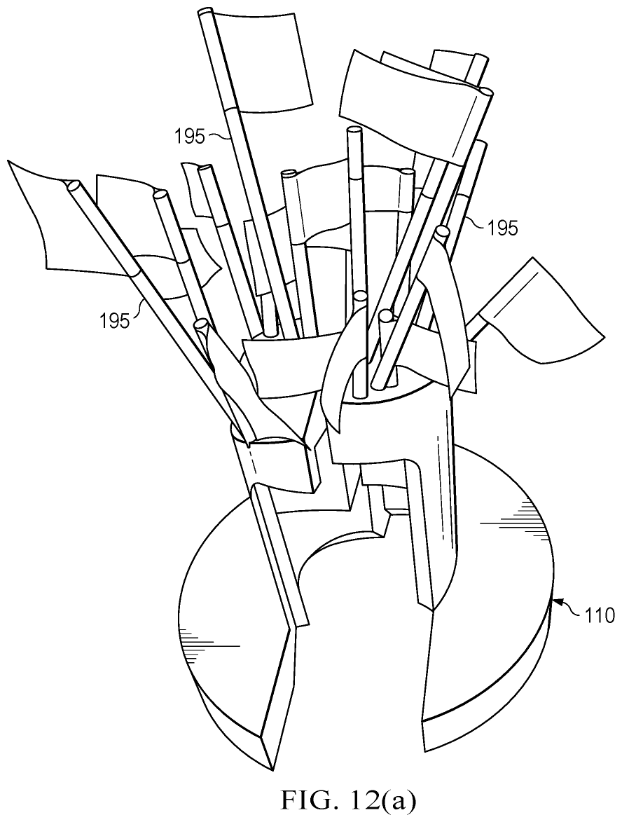

Referring to FIGS. 11(a)-(c), with continuing reference to FIGS. 10(c), in one or more embodiments, the dipoles 120 are wired to a junction box 190 in order to expose the different contacts of the dipoles 120 for ease of connection to different instruments.

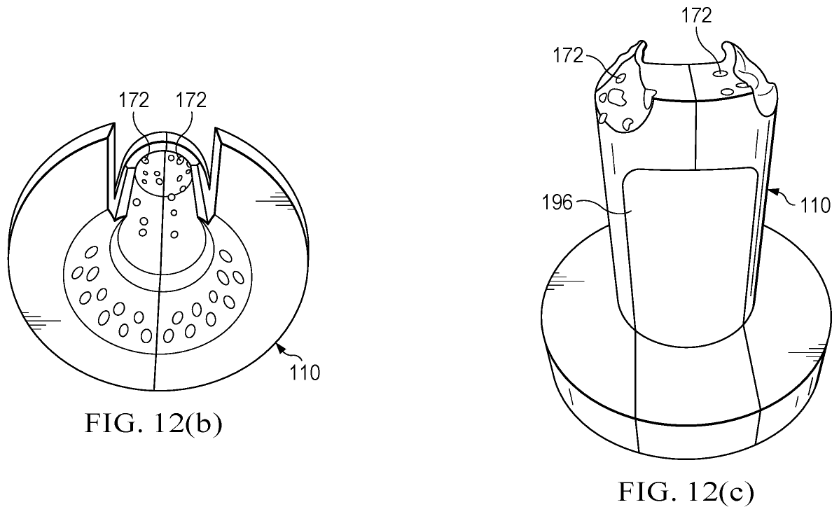

Referring to FIGS. 12(a)-(c), with continuing reference to FIG. 7(c), in one or more embodiments, the dipoles 120 are inserted through the base 110 after pouring the simulated brain layer 115. At the first step, empty CF tubes 195 are installed. These tubes 195 are purposely clogged at the tip with a small amount of clay to prevent the silicone from leaking into the tubes 195. Once the Si-Ca Mix 165 of the simulated brain layer 115 is at least partially cured, thinner rods carrying the dipoles 120 are inserted through the guides 195, penetrating into the simulated brain layer 115's mesh of CF. To ensure a correct placement, the length of each guide tube 195 is 5 mm shorter than its corresponding dipole rod, ensuring a minimum required penetration for the dipole 120. To ease insertion of the dipoles 120 by accommodating for their unpredictable angles of insertion, in one or more embodiments, the base 110 includes a removable part 196, providing clearance for insertion of the dipole rods, as shown in FIG. 12(c). Accurate labeling of the holes 172 is important to prevent losing track of which dipole 120 corresponds to which hole 172.

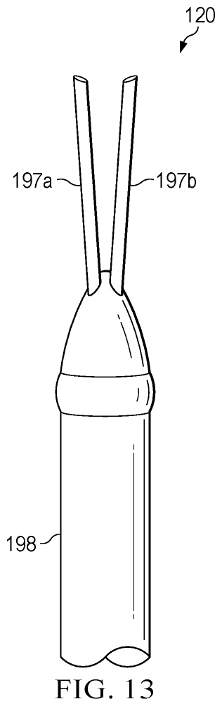

Referring to FIG. 13, with continuing reference to FIGS. 12(a)-(c), in one or more embodiments, each dipole 120 includes a pair of dipole wires 197a-b extending through, and exposed proximate an end of, a CF dipole tube 198. This “single rod” design facilitates insertion of the dipole 120 through the guide tube 195 with enough clearance to allow the dipole wires 197a-b to travel to the tip and remain twisted all the way until reaching the end. However, care must be taken to ensure that the dipole wires 197a-b—at the tip do not converge or diverge, losing their parallelism while penetrating into the simulated brain layer 115.

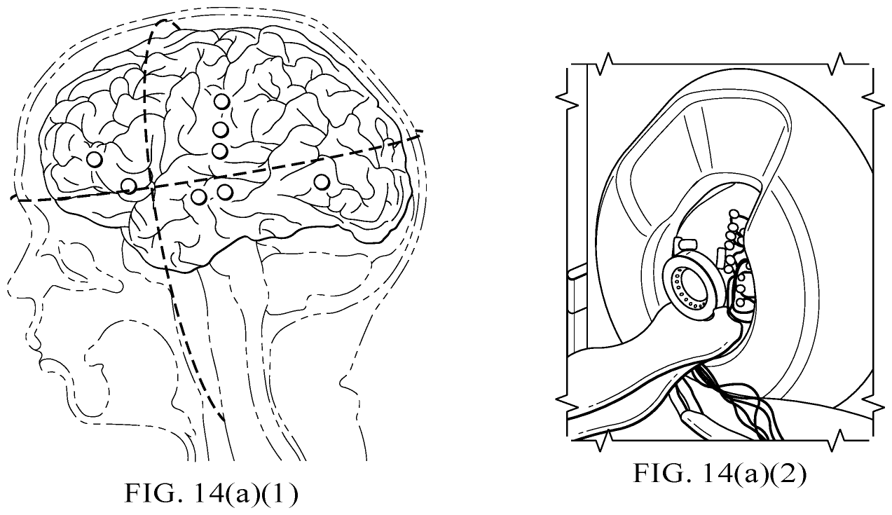

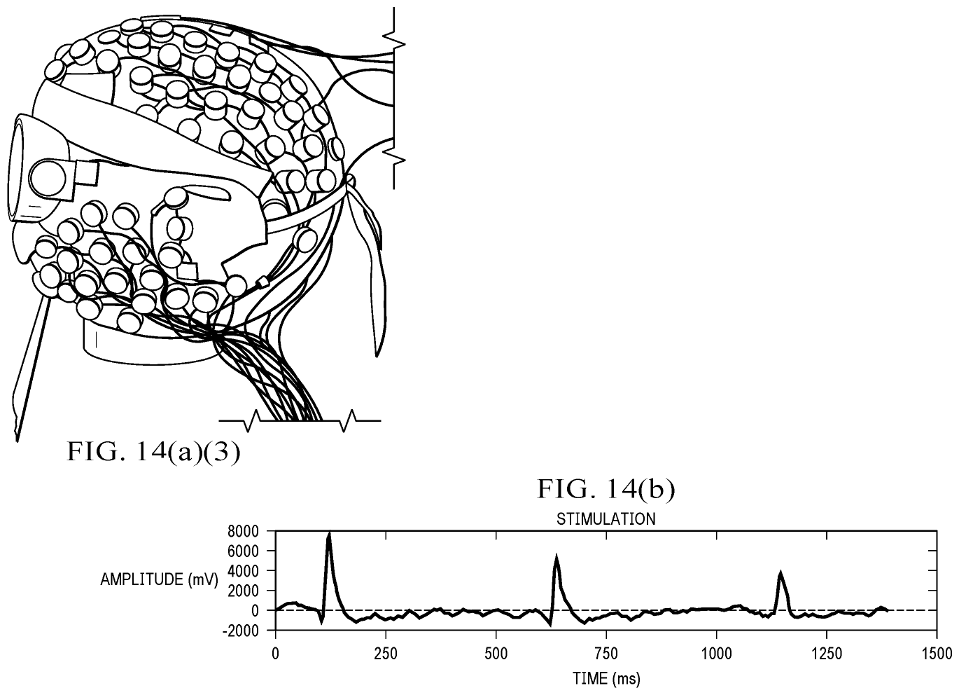

Referring to FIGS. 14(a)(1)-(a)(3), in one or more embodiments, simultaneous MEG and HD-EEG (e.g., 1 kHz sampling rate) are recorded while the dipolar sources 120 of the phantom 105 are stimulated using a waveform generator. Turning also to FIG. 14(b), with continuing reference to FIGS. 14(a)(1)-(a)(3), in one or more embodiments, the signal that stimulates the dipolar sources 120 of the phantom 105 is obtained, for example, from an intracranial EEG (“iEEG”) recording of the left temporal lobe of a male, 17-year-old, drug resistant epilepsy patient, and is band-pass filtered between 1 and 70 Hz to include interictal epileptiform discharges (“IEDs”) over a time span of 1.4 seconds. This signal is then regenerated and fed to the implanted dipolar sources 120 using the waveform generator (e.g., RIGOL DG-1032z) with an output sampling frequency of 2 kHz.

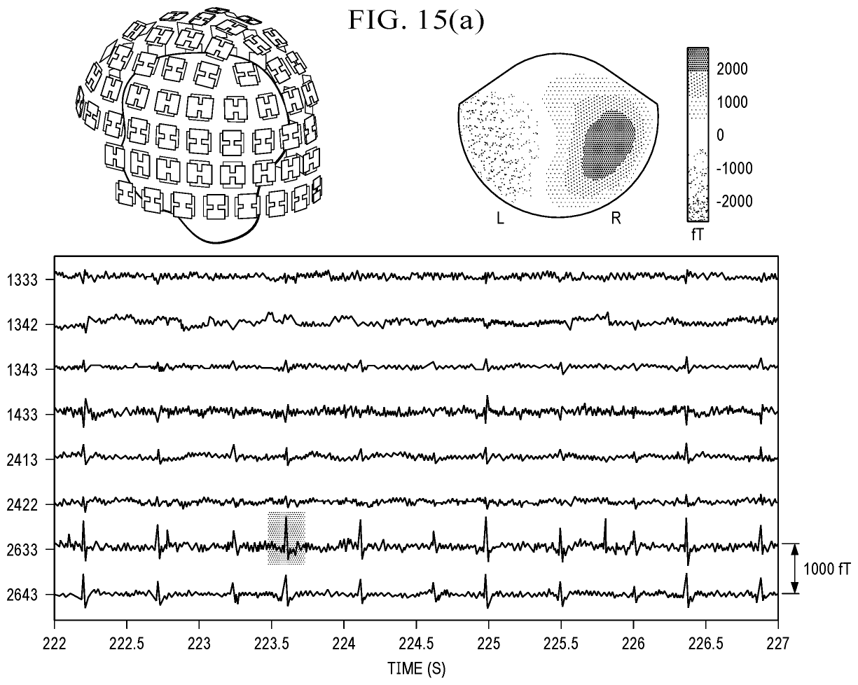

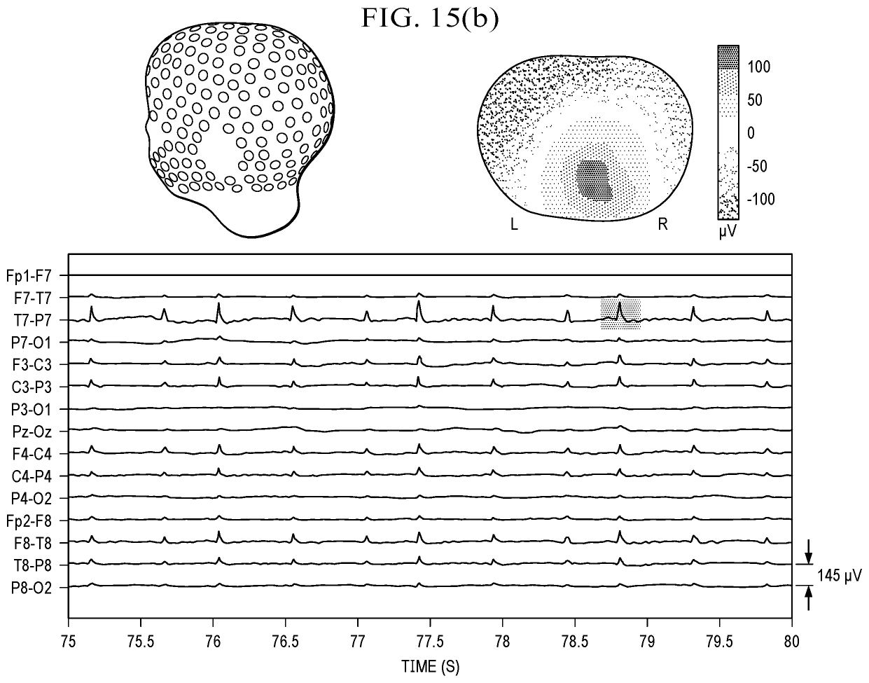

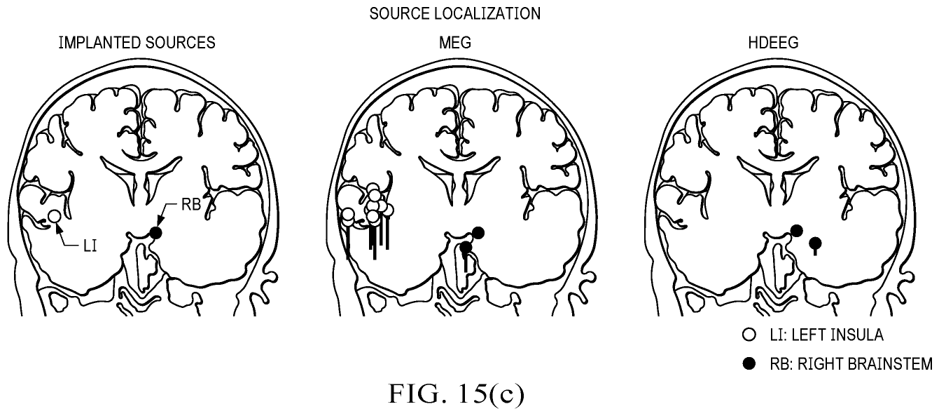

Referring to FIGS. 15(a)-(c), with continuing reference to FIGS. 14(a)(1)-(b), in one or more embodiments, the locations of the implanted dipolar sources 120 are marked using a CT scan of the phantom 105, and source localization is performed at the peak of each spike using equivalent current dipole (ECD) (Goodness of Fit >90%) via Brainstorm. As a result, Realistic IEDs in terms of amplitude, duration, and morphology were obtained in both MEG and HD-EEG recordings, as shown in FIGS. 15(a)-(b). For MEG data, two dipolar sources 120 positioned proximate to the left insula and right brainstem had a localization error of 8.6±0 mm and 8.6±2.9 mm, respectively, as shown in FIG. 15(c). Likewise, for HD-EEG data, the dipolar source 120—positioned proximate to the right brainstem had a localization error of 11.4±0 mm, as shown in FIG. 15(c).

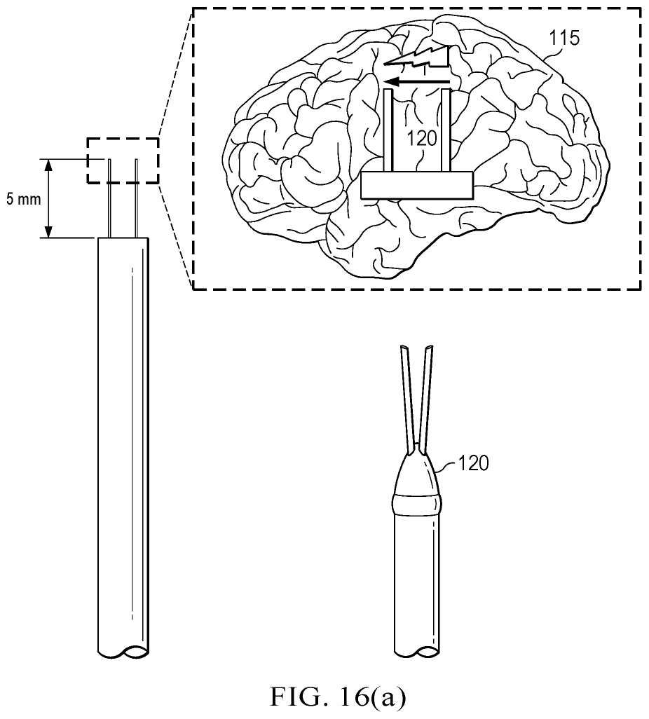

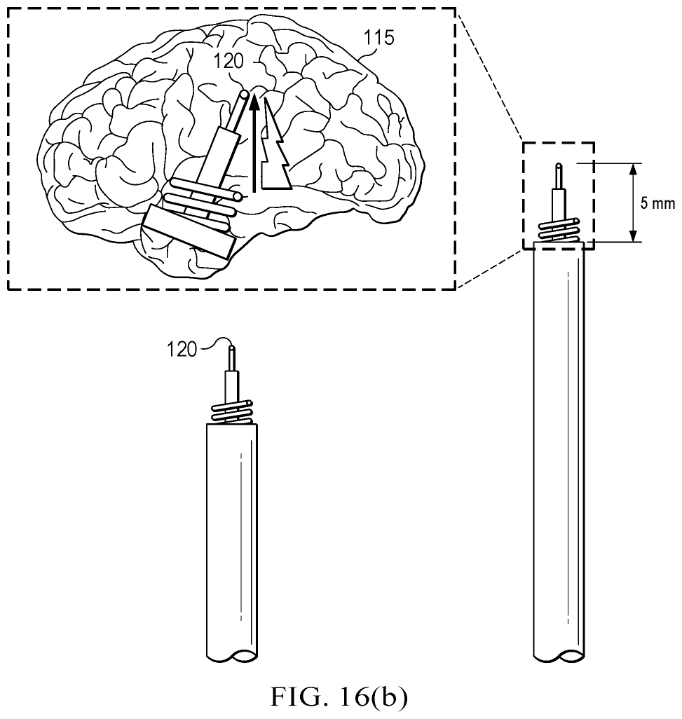

Referring to FIGS. 16(a)-(b), with continuing reference to FIG. 13, in one or more embodiments, the dipolar sources 120 include tangential and/or radial source(s). The orientation of the neurons that fire electrical signals in the brain plays an important role in the detectability of the signal, especially in MEG recording systems. In light of this feature, in one or more embodiments, the phantom 105 includes one or more tangential dipolar sources at pre-defined lobe(s) in one hemisphere of the simulated brain 115, the other hemisphere of the simulated brain 115, or both. Additionally, or alternatively, in one or more embodiments, the phantom 105 includes one or more radial dipolar sources in the one hemisphere of the brain, the other hemisphere of the brain, or both. The tangential and/or radial dipolar source(s) allow different combinations of source depth and orientation, which enhances the resemblance of the dipolar sources 120 to that of real brain sources. Furthermore, the tangential and/or radial dipolar source(s) facilitate the study of various types of brain sources in evaluating the quality of EEG and MEG recordings and/or the accuracy of corresponding source localization methods.

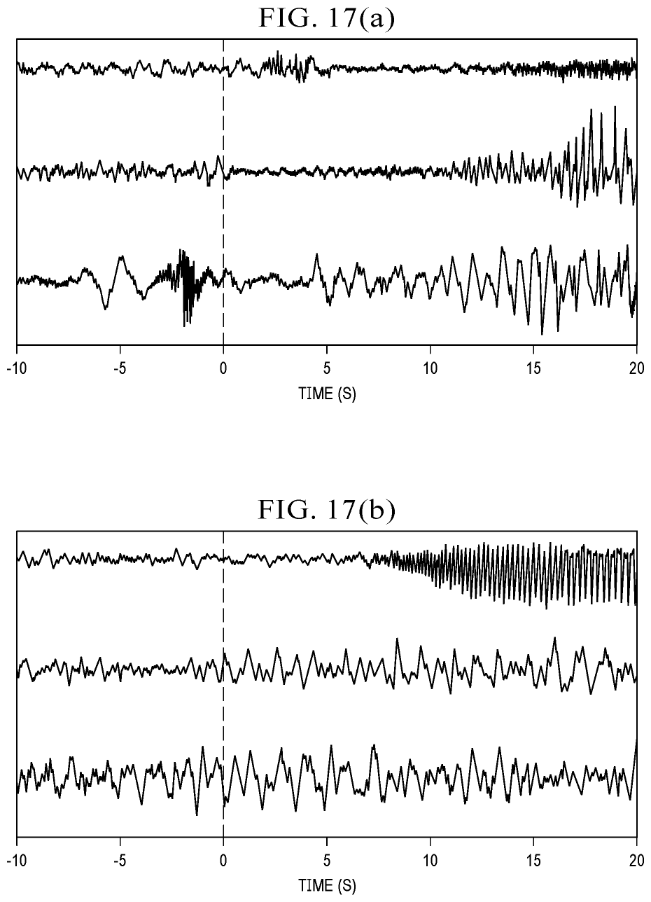

Referring to FIGS. 17(a)-(b), with continuing reference to FIG. 14(b), in one or more embodiments, in addition to, or instead of, stimulating the dipolar sources 120 with interictal spike epochs taken from a medically refractory epilepsy patient (as described above in connection with FIG. 14(b)), the dipolar sources 120 may be stimulated with other types of epilepsy biomarkers, such as a fast seizure onset (shown in FIG. 17(a)), a slow seizure onset (shown in FIG. 17(b)), interictal ripples, fast ripples, the like, or any combination thereof, thus providing a robust ground truth for assessment of commercial and academic algorithms for epileptic biomarker and seizure detection.

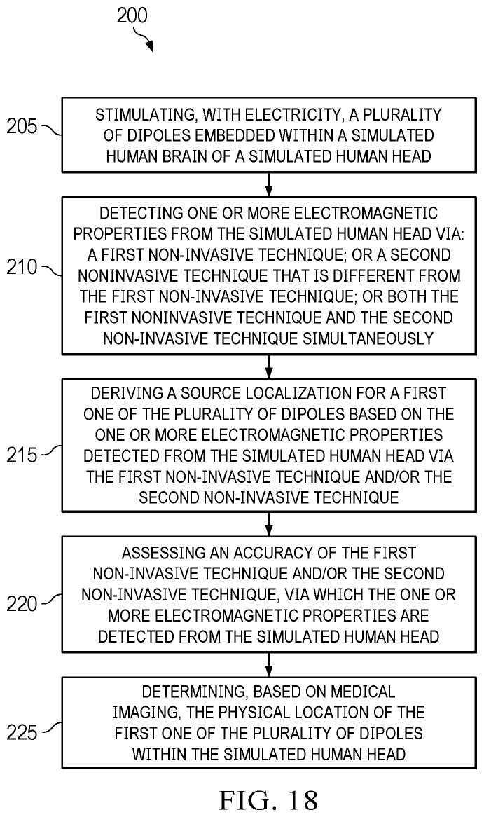

Referring to FIG. 18, with continuing reference to FIGS. 1(a) through 17, a method 200 is illustrated according to one or more embodiments of the present disclosure. At a step 205 of the method 200, a plurality of dipoles (120) embedded within a simulated human brain (115) of a simulated human head (105) are stimulated with electricity. In one or more embodiments, the electricity with which the plurality of dipoles (120) are stimulated is based on a recording of an animate human head. In one or more embodiments, at another step of the method 200, said recording of the animate human head is captured. In one or more embodiments, the animate human head is that of a drug resistant epilepsy patient. In one or more embodiments stimulating the plurality of dipoles (120) with the electricity causes the simulated human head (105) to generate one or more electromagnetic properties that simulate same of the animate human head. At yet another step 210 of the method 200, the one or more electromagnetic properties are detected from the simulated human head (105) via: a first non-invasive technique (e.g., implemented using the first non-invasive brain activity recording system 140); or a second non-invasive technique (e.g., implemented using the first non-invasive brain activity recording system 140) that is different from the first non-invasive technique; or both the first non-invasive technique and the second non-invasive technique simultaneously. In one or more embodiments, the first non-invasive technique is, or includes, electroencephalography (“EEG”), and the second non-invasive technique is, or includes, magnetoencephalography (“MEG”). In one or more embodiments, the one or more electromagnetic properties are detected via both the first and second non-invasive techniques simultaneously. At yet another step 215 of the method 200, a source localization is derived for a first one of the plurality of dipoles (120) based on the one or more electromagnetic properties detected from the simulated human head (105) via the first non-invasive technique and/or the second non-invasive technique. At yet another step 220 of the method 200, an accuracy of the first non-invasive technique and/or the second non-invasive technique, via which the one or more electromagnetic properties are detected from the simulated human head (105), is assessed. In one or more embodiments, assessing the accuracy of the first non-invasive technique and/or the second non-invasive technique includes comparing the derived source localization for the first one of the plurality of dipoles (120) with a physical location of the first one of the plurality of dipoles (120) within the simulated human head (105). At yet another step 225 of the method 200, the physical location of the first one of the plurality of dipoles (120) within the simulated human head (105) is determined based on medical imaging (e.g., acquired using the non-invasive imaging device/system 150). In one or more embodiments of the method 200, a first one of the plurality of dipoles (120) is oriented tangentially within the simulated human brain (115), and a second one of the plurality of dipoles (120) is oriented radially within the simulated human brain (115).

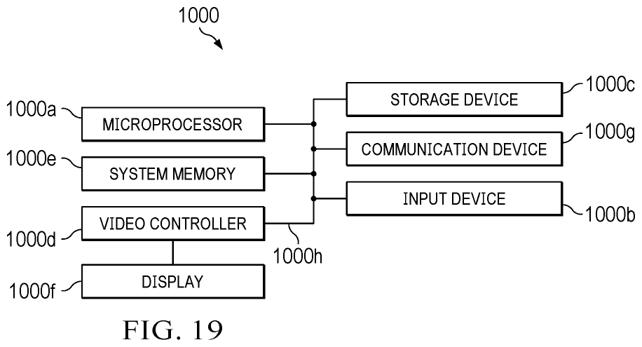

Referring to FIG. 19, with continuing reference to FIGS. 1(a) through 18, in one or more embodiments, a computing node 1000 for implementing one or more of the above-described embodiments, and/or any combination thereof, is depicted. In one or more embodiments, the node 1000 is, includes, or is part of the controller 135 shown and described above in connection with FIG. 1. The node 1000 includes a microprocessor 1000a, an input device 1000b, a storage device 1000c, a video controller 1000d, a system memory 1000e, a display 1000f, and a communication device 1000g all interconnected by one or more buses 1000h. In one or more embodiments, the microprocessor 1000a is, includes, or is part of, the phantom 105 and/or the instruments described herein. In one or more embodiments, the storage device 1000c may include a floppy drive, hard drive, CD-ROM, optical drive, any other form of storage device or any combination thereof. In one or more embodiments, the storage device 1000c may include, and/or be capable of receiving, a floppy disk, CD-ROM, DVD-ROM, or any other form of computer-readable medium that may contain executable instructions. In one or more embodiments, the communication device 1000g may include a modem, network card, or any other device to enable the node 1000 to communicate with other nodes. In one or more embodiments, any node represents a plurality of interconnected (whether by intranet or Internet) computer systems, including without limitation, personal computers, mainframes, PDAs, smartphones and cell phones.

In one or more embodiments, one or more of the components of any of the above-described embodiments include at least the node 1000 and/or components thereof, and/or one or more nodes that are substantially similar to the node 1000 and/or components thereof. In one or more embodiments, one or more of the above-described components of the node 1000 and/or the above-described embodiments include respective pluralities of same components.

In one or more embodiments, a computer system includes at least hardware capable of executing machine readable instructions, as well as the software for executing acts (typically machine-readable instructions) that produce a desired result. In one or more embodiments, a computer system includes hybrids of hardware and software, as well as computer sub-systems.

In one or more embodiments, hardware generally includes at least processor-capable platforms, such as client-machines (also known as personal computers or servers), and hand-held processing devices (such as smart phones, tablet computers, personal digital assistants (PDAs), or personal computing devices (PCDs), for example). In one or more embodiments, hardware may include any physical device that is capable of storing machine-readable instructions, such as memory or other data storage devices. In one or more embodiments, other forms of hardware include hardware sub-systems, including transfer devices such as modems, modem cards, ports, and port cards, for example.

In one or more embodiments, software includes any machine code stored in any memory medium, such as RAM or ROM, and machine code stored on other devices (such as floppy disks, flash memory, or a CD ROM, for example). In one or more embodiments, software may include source or object code. In one or more embodiments, software encompasses any set of instructions capable of being executed on a node such as, for example, on a client machine or server.

In one or more embodiments, combinations of software and hardware could also be used for providing enhanced functionality and performance for certain embodiments of the present disclosure. In one or more embodiments, software functions may be directly manufactured into a silicon chip. Accordingly, combinations of hardware and software are also included within the definition of a computer system and are thus envisioned by the present disclosure as possible equivalent structures and equivalent methods.

In one or more embodiments, computer readable mediums include, for example, passive data storage, such as a random-access memory (RAM) as well as semi-permanent data storage such as a compact disk read only memory (CD-ROM). One or more embodiments of the present disclosure may be embodied in the RAM of a computer to transform a standard computer into a new specific computing machine. In one or more embodiments, data structures are defined organizations of data that may enable one or more embodiments of the present disclosure. In one or more embodiments, data structure may provide an organization of data, or an organization of executable code.

In one or more embodiments, any networks and/or one or more portions thereof, may be designed to work on any specific architecture. In one or more embodiments, one or more portions of any networks may be executed on a single computer, local area networks, client-server networks, wide area networks, internets, hand-held and other portable and wireless devices and networks.

In one or more embodiments, database may be any standard or proprietary database software. In one or more embodiments, the database may have fields, records, data, and other database elements that may be associated through database specific software. In one or more embodiments, data may be mapped. In one or more embodiments, mapping is the process of associating one data entry with another data entry. In one or more embodiments, the data contained in the location of a character file can be mapped to a field in a second table. In one or more embodiments, the physical location of the database is not limiting, and the database may be distributed. In one or more embodiments, the database may exist remotely from the server, and run on a separate platform. In one or more embodiments, the database may be accessible across the Internet. In one or more embodiments, more than one database may be implemented.

In one or more embodiments, a plurality of instructions stored on a non-transitory computer readable medium may be executed by one or more processors to cause the one or more processors to carry out or implement in whole or in part the above-described operation of each of the above-described embodiments, and/or any combination thereof. In one or more embodiments, such a processor may be or include one or more of the microprocessor 1000a, one or more other controllers, any processor(s) that are part of the components of the above-described embodiments, and/or any combination thereof, and such a computer readable medium may be distributed among one or more components of the above-described systems. In one or more embodiments, such a processor may execute the plurality of instructions in connection with a virtual computer system. In one or more embodiments, such a plurality of instructions may communicate directly with the one or more processors, and/or may interact with one or more operating systems, middleware, firmware, other applications, and/or any combination thereof, to cause the one or more processors to execute the instructions.

One or more embodiments of the present application are provided in whole or in part as described in the '606 Application, in the Appendix of the '606 Application, in the '677 Application, and in the Appendix of the '677 Application. It is understood that one or more of the embodiments described above and shown FIGS. 1(a) through 19 may be combined in whole or in part with one or more of the embodiments described and illustrated in the '606 Application, one or more of the embodiments described and illustrated in the Appendix of the '606 Application, one or more of the embodiments described and illustrated in the '677 Application, one or more of the embodiments described and illustrated in the Appendix of the '677 Application, and/or one or more of the other embodiments described above and shown in FIGS. 1(a) through 19.

A first method has been disclosed. The first method generally includes: stimulating, with electricity, a plurality of dipoles embedded within a simulated human brain of a simulated human head, wherein stimulating the plurality of dipoles with the electricity causes the simulated human head to generate one or more electromagnetic properties that simulate same of an animate human head; and detecting the one or more electromagnetic properties from the simulated human head via both: a first non-invasive technique; and a second non-invasive technique that is different from the first non-invasive technique, wherein the one or more electromagnetic properties are detected via both the first and second non-invasive techniques simultaneously. In one or more embodiments: the first non-invasive technique is, or includes, electroencephalography (“EEG”); and the second non-invasive technique is, or includes, magnetoencephalography (“MEG”). In one or more embodiments, the first method further includes deriving a source localization for a first one of the plurality of dipoles based on the one or more electromagnetic properties detected from the simulated human head via both the first non-invasive technique and the second non-invasive technique. In one or more embodiments, the first method further includes assessing an accuracy of both the first non-invasive technique and the second non-invasive technique, via which the one or more electromagnetic properties are detected from the simulated human head; wherein assessing the accuracy of both the first non-invasive technique and the second non-invasive technique includes comparing the derived source localization for the first one of the plurality of dipoles with a physical location of the first one of the plurality of dipoles within the simulated human head. In one or more embodiments, the first method further includes determining, based on medical imaging, the physical location of the first one of the plurality of dipoles within the simulated human head. In one or more embodiments, the electricity with which the plurality of dipoles are stimulated is based on a recording of the animate human head. In one or more embodiments, the first method further includes capturing said recording of the animate human head. In one or more embodiments, the animate human head is that of a drug resistant epilepsy patient. In one or more embodiments, the electricity with which the plurality of dipoles are stimulated is based on recordings of a plurality of animate human heads. In one or more embodiments: a first one of the plurality of dipoles is oriented tangentially within the simulated human brain; and a second one of the plurality of dipoles is oriented radially within the simulated human brain.

A second method has also been disclosed. The second method generally includes: stimulating, with electricity, a plurality of dipoles embedded within a simulated human brain of a simulated human head, wherein the electricity with which the plurality of dipoles are stimulated is based on a recording of an animate human head, and wherein stimulating the plurality of dipoles with the electricity causes the simulated human head to generate one or more electromagnetic properties that simulate same of the animate human head; and detecting the one or more electromagnetic properties from the simulated human head via: a first non-invasive technique; or a second non-invasive technique that is different from the first non-invasive technique; or both the first non-invasive technique and the second non-invasive technique simultaneously. In one or more embodiments, the second method further includes capturing said recording of the animate human head. In one or more embodiments, the animate human head is that of a drug resistant epilepsy patient. In one or more embodiments: the first non-invasive technique is, or includes, electroencephalography (“EEG”); and the second non-invasive technique is, or includes, magnetoencephalography (“MEG”). In one or more embodiments, the one or more electromagnetic properties are detected via both the first and second non-invasive techniques simultaneously. In one or more embodiments, the second method further includes deriving a source localization for a first one of the plurality of dipoles based on the one or more electromagnetic properties detected from the simulated human head via the first non-invasive technique and/or the second non-invasive technique. In one or more embodiments, the second method further includes assessing an accuracy of the first non-invasive technique and/or the second non-invasive technique, via which the one or more electromagnetic properties are detected from the simulated human head; wherein assessing the accuracy of the first non-invasive technique and/or the second non-invasive technique includes comparing the derived source localization for the first one of the plurality of dipoles with a physical location of the first one of the plurality of dipoles within the simulated human head. In one or more embodiments, the second method further includes determining, based on medical imaging, the physical location of the first one of the plurality of dipoles within the simulated human head. In one or more embodiments: a first one of the plurality of dipoles is oriented tangentially within the simulated human brain; and a second one of the plurality of dipoles is oriented radially within the simulated human brain.

A first system has also been disclosed. The first system generally includes: a non-transitory computer readable medium; and a plurality of instructions stored on the non-transitory computer readable medium and executable by one or more processors; wherein the instructions are executed by the one or more processors so that the following steps are executed: stimulating, with electricity, a plurality of dipoles embedded within a simulated human brain of a simulated human head, wherein stimulating the plurality of dipoles with the electricity causes the simulated human head to generate one or more electromagnetic properties that simulate same of an animate human head; and detecting the one or more electromagnetic properties from the simulated human head via both: a first non-invasive technique; and a second non-invasive technique that is different from the first non-invasive technique, wherein the one or more electromagnetic properties are detected via both the first and second non-invasive techniques simultaneously. In one or more embodiments: the first non-invasive technique is, or includes, electroencephalography (“EEG”); and the second non-invasive technique is, or includes, magnetoencephalography (“MEG”). In one or more embodiments, the instructions are executed by the one or more processors so that the following step is also executed: deriving a source localization for a first one of the plurality of dipoles based on the one or more electromagnetic properties detected from the simulated human head via both the first non-invasive technique and the second non-invasive technique. In one or more embodiments, the instructions are executed by the one or more processors so that the following step is also executed: assessing an accuracy of both the first non-invasive technique and the second non-invasive technique, via which the one or more electromagnetic properties are detected from the simulated human head; wherein assessing the accuracy of both the first non-invasive technique and the second non-invasive technique includes comparing the derived source localization for the first one of the plurality of dipoles with a physical location of the first one of the plurality of dipoles within the simulated human head. In one or more embodiments, the instructions are executed by the one or more processors so that the following step is also executed: determining, based on medical imaging, the physical location of the first one of the plurality of dipoles within the simulated human head. In one or more embodiments, the electricity with which the plurality of dipoles are stimulated is based on a recording of the animate human head. In one or more embodiments, the instructions are executed by the one or more processors so that the following step is also executed: capturing said recording of the animate human head. In one or more embodiments, the animate human head is that of a drug resistant epilepsy patient. In one or more embodiments, the electricity with which the plurality of dipoles are stimulated is based on recordings of a plurality of animate human heads. In one or more embodiments, the first system further includes: the simulated human brain; and first and second ones of the plurality of dipoles embedded in the simulated human brain; wherein: the first one of the plurality of dipoles is oriented tangentially within the simulated human brain; and the second one of the plurality of dipoles is oriented radially within the simulated human brain.

A second system has also been disclosed. The second system generally includes: a non-transitory computer readable medium; and a plurality of instructions stored on the non-transitory computer readable medium and executable by one or more processors; wherein the instructions are executed by the one or more processors so that the following steps are executed: stimulating, with electricity, a plurality of dipoles embedded within a simulated human brain of a simulated human head, wherein the electricity with which the plurality of dipoles are stimulated is based on a recording of an animate human head, and wherein stimulating the plurality of dipoles with the electricity causes the simulated human head to generate one or more electromagnetic properties that simulate same of the animate human head; and detecting the one or more electromagnetic properties from the simulated human head via: a first non-invasive technique; or a second non-invasive technique that is different from the first non-invasive technique; or both the first non-invasive technique and the second non-invasive technique simultaneously. In one or more embodiments, the instructions are executed by the one or more processors so that the following step is also executed: capturing said recording of the animate human head. In one or more embodiments, the animate human head is that of a drug resistant epilepsy patient. In one or more embodiments: the first non-invasive technique is, or includes, electroencephalography (“EEG”); and the second non-invasive technique is, or includes, magnetoencephalography (“MEG”). In one or more embodiments, the one or more electromagnetic properties are detected via both the first and second non-invasive techniques simultaneously. In one or more embodiments, the instructions are executed by the one or more processors so that the following step is also executed: deriving a source localization for a first one of the plurality of dipoles based on the one or more electromagnetic properties detected from the simulated human head via the first non-invasive technique and/or the second non-invasive technique. In one or more embodiments, the instructions are executed by the one or more processors so that the following step is also executed: assessing an accuracy of the first non-invasive technique and/or the second non-invasive technique, via which the one or more electromagnetic properties are detected from the simulated human head; wherein assessing the accuracy of the first non-invasive technique and/or the second non-invasive technique includes comparing the derived source localization for the first one of the plurality of dipoles with a physical location of the first one of the plurality of dipoles within the simulated human head. In one or more embodiments, the instructions are executed by the one or more processors so that the following step is also executed: determining, based on medical imaging, the physical location of the first one of the plurality of dipoles within the simulated human head. In one or more embodiments, the second system further includes: the simulated human brain; and first and second ones of the plurality of dipoles embedded in the simulated human brain; wherein: the first one of the plurality of dipoles is oriented tangentially within the simulated human brain; and the second one of the plurality of dipoles is oriented radially within the simulated human brain.

It is further understood that variations may be made in the foregoing without departing from the scope of the disclosure.

In one or more embodiments, the elements and teachings of the various embodiments disclosed herein may be combined in whole or in part in some or all of said embodiment(s). In addition, one or more of the elements and teachings of the various embodiments disclosed herein may be omitted, at least in part, or combined, at least in part, with one or more of the other elements and teachings of said embodiment(s).

Any spatial references such as, for example, “upper,” “lower,” “above,” “below,” “between,” “bottom,” “vertical,” “horizontal,” “angular,” “upwards,” “downwards,” “side-to-side,” “left-to-right,” “left,” “right,” “right-to-left,” “top-to-bottom,” “bottom-to-top,” “top,” “bottom,” “bottom-up,” “top-down,” etc., are for the purpose of illustration only and do not limit the specific orientation or location of the structure described above.

In one or more embodiments, while different steps, processes, and procedures are described as appearing as distinct acts, one or more of the steps, one or more of the processes, or one or more of the procedures may also be performed in different orders, simultaneously or sequentially. In one or more embodiments, the steps, processes or procedures may be merged into one or more steps, processes or procedures. In one or more embodiments, one or more of the operational steps in each embodiment may be omitted. Moreover, in some instances, some features of the present disclosure may be employed without a corresponding use of the other features. Moreover, one or more of the embodiments disclosed above, in the '606 Application, in the Appendix of the '606 Application, in the '677 Application, and in the Appendix of the '677 Application, or variations thereof, may be combined in whole or in part with any one or more of the other embodiments described above, in the '606 Application, in the Appendix of the '606 Application, in the '677 Application, and in the Appendix of the '677 Application, or variations thereof.

Although various embodiments have been disclosed in detail above, in the '606 Application, in the Appendix of the '606 Application, in the '677 Application, and in the Appendix of the '677 Application, the embodiments disclosed are exemplary only and are not limiting, and those skilled in the art will readily appreciate that many other modifications, changes, and substitutions are possible in the embodiments without materially departing from the novel teachings and advantages of the present disclosure. Accordingly, all such modifications, changes, and substitutions are intended to be included within the scope of this disclosure as defined in the following claims. In the claims, means-plus-function clauses are intended to cover the structures described herein as performing the recited function and not only structural equivalents, but also equivalent structures. Moreover, it is the express intention of the applicant not to invoke 35 U.S.C. § 112 (f) for any limitations of any of the claims herein, except for those in which the claim expressly uses the word “means” together with an associated function.

What is claimed is:

1. A method, comprising:
stimulating, with electricity, a plurality of dipoles embedded within a simulated human brain of a simulated human head,
wherein stimulating the plurality of dipoles with the electricity causes the simulated human head to generate one or more electromagnetic properties that simulate same of an animate human head;
and
detecting the one or more electromagnetic properties from the simulated human head via both:
a first non-invasive technique; and
a second non-invasive technique that is different from the first non-invasive technique,
wherein the one or more electromagnetic properties are detected via both the first and second non-invasive techniques simultaneously.

2. The method of claim 1, wherein:
the first non-invasive technique is, or includes, electroencephalography (“EEG”); and
the second non-invasive technique is, or includes, magnetoencephalography (“MEG”).

3. The method of claim 1, further comprising:
deriving a source localization for a first one of the plurality of dipoles based on the one or more electromagnetic properties detected from the simulated human head via both the first non-invasive technique and the second non-invasive technique.

4. The method of claim 3, further comprising:
assessing an accuracy of both the first non-invasive technique and the second non-invasive technique, via which the one or more electromagnetic properties are detected from the simulated human head;
wherein assessing the accuracy of both the first non-invasive technique and the second non-invasive technique comprises:
comparing the derived source localization for the first one of the plurality of dipoles with a physical location of the first one of the plurality of dipoles within the simulated human head.

5. The method of claim 4, further comprising:
determining, based on medical imaging, the physical location of the first one of the plurality of dipoles within the simulated human head.

6. The method of claim 1, wherein the electricity with which the plurality of dipoles are stimulated is based on a recording of the animate human head.

7. The method of claim 6, further comprising:
capturing said recording of the animate human head.

8. The method of claim 6, wherein the animate human head is that of a drug resistant epilepsy patient.

9. The method of claim 1, wherein the electricity with which the plurality of dipoles are stimulated is based on recordings of a plurality of animate human heads.

10. The method of claim 1, wherein:
a first one of the plurality of dipoles is oriented tangentially within the simulated human brain; and
a second one of the plurality of dipoles is oriented radially within the simulated human brain.

11. A method, comprising:
stimulating, with electricity, a plurality of dipoles embedded within a simulated human brain of a simulated human head,
wherein the electricity with which the plurality of dipoles are stimulated is based on a recording of an animate human head, and
wherein stimulating the plurality of dipoles with the electricity causes the simulated human head to generate one or more electromagnetic properties that simulate same of the animate human head;
and
detecting the one or more electromagnetic properties from the simulated human head via:
a first non-invasive technique; or
a second non-invasive technique that is different from the first non-invasive technique; or
both the first non-invasive technique and the second non-invasive technique simultaneously.

12. The method of claim 11, further comprising:
capturing said recording of the animate human head.

13. The method of claim 11, wherein the animate human head is that of a drug resistant epilepsy patient.

14. The method of claim 11, wherein:
the first non-invasive technique is, or includes, electroencephalography (“EEG”); and
the second non-invasive technique is, or includes, magnetoencephalography (“MEG”).

15. The method of claim 11, wherein the one or more electromagnetic properties are detected via both the first and second non-invasive techniques simultaneously.

16. The method of claim 11, further comprising:
deriving a source localization for a first one of the plurality of dipoles based on the one or more electromagnetic properties detected from the simulated human head via the first non-invasive technique and/or the second non-invasive technique.

17. The method of claim 16, further comprising:
assessing an accuracy of the first non-invasive technique and/or the second non-invasive technique, via which the one or more electromagnetic properties are detected from the simulated human head;
wherein assessing the accuracy of the first non-invasive technique and/or the second non-invasive technique comprises:
comparing the derived source localization for the first one of the plurality of dipoles with a physical location of the first one of the plurality of dipoles within the simulated human head.

18. The method of claim 17, further comprising:
determining, based on medical imaging, the physical location of the first one of the plurality of dipoles within the simulated human head.

19. The method of claim 11, wherein:
a first one of the plurality of dipoles is oriented tangentially within the simulated human brain; and
a second one of the plurality of dipoles is oriented radially within the simulated human brain.

20. A system, comprising:
a non-transitory computer readable medium; and
a plurality of instructions stored on the non-transitory computer readable medium and executable by one or more processors;
wherein the instructions are executed by the one or more processors so that the following steps are executed:
stimulating, with electricity, a plurality of dipoles embedded within a simulated human brain of a simulated human head,
wherein stimulating the plurality of dipoles with the electricity causes the simulated human head to generate one or more electromagnetic properties that simulate same of an animate human head;
and
detecting the one or more electromagnetic properties from the simulated human head via both:
a first non-invasive technique; and
a second non-invasive technique that is different from the first non-invasive technique,
wherein the one or more electromagnetic properties are detected via both the first and second non-invasive techniques simultaneously.

21. The system of claim 20, wherein:
the first non-invasive technique is, or includes, electroencephalography (“EEG”); and
the second non-invasive technique is, or includes, magnetoencephalography (“MEG”).

22. The system of claim 20, wherein the instructions are executed by the one or more processors so that the following step is also executed:
deriving a source localization for a first one of the plurality of dipoles based on the one or more electromagnetic properties detected from the simulated human head via both the first non-invasive technique and the second non-invasive technique.

23. The system of claim 22, wherein the instructions are executed by the one or more processors so that the following step is also executed:
assessing an accuracy of both the first non-invasive technique and the second non-invasive technique, via which the one or more electromagnetic properties are detected from the simulated human head;
wherein assessing the accuracy of both the first non-invasive technique and the second non-invasive technique comprises:
comparing the derived source localization for the first one of the plurality of dipoles with a physical location of the first one of the plurality of dipoles within the simulated human head.

24. The system of claim 23, wherein the instructions are executed by the one or more processors so that the following step is also executed:
determining, based on medical imaging, the physical location of the first one of the plurality of dipoles within the simulated human head.

25. The system of claim 20, wherein the electricity with which the plurality of dipoles are stimulated is based on a recording of the animate human head.

26. The system of claim 25, wherein the instructions are executed by the one or more processors so that the following step is also executed:
capturing said recording of the animate human head.

27. The system of claim 25, wherein the animate human head is that of a drug resistant epilepsy patient.

28. The system of claim 20, wherein the electricity with which the plurality of dipoles are stimulated is based on recordings of a plurality of animate human heads.

29. The system of claim 20, further comprising:
the simulated human brain; and
first and second ones of the plurality of dipoles embedded in the simulated human brain;
wherein:
the first one of the plurality of dipoles is oriented tangentially within the simulated human brain; and
the second one of the plurality of dipoles is oriented radially within the simulated human brain.

30. A system, comprising:
a non-transitory computer readable medium; and
a plurality of instructions stored on the non-transitory computer readable medium and executable by one or more processors;
wherein the instructions are executed by the one or more processors so that the following steps are executed:
stimulating, with electricity, a plurality of dipoles embedded within a simulated human brain of a simulated human head,
wherein the electricity with which the plurality of dipoles are stimulated is based on a recording of an animate human head, and
wherein stimulating the plurality of dipoles with the electricity causes the simulated human head to generate one or more electromagnetic properties that simulate same of the animate human head;
and
detecting the one or more electromagnetic properties from the simulated human head via:
a first non-invasive technique; or
a second non-invasive technique that is different from the first non-invasive technique; or
both the first non-invasive technique and the second non-invasive technique simultaneously.

31. The system of claim 30, wherein the instructions are executed by the one or more processors so that the following step is also executed:
capturing said recording of the animate human head.

32. The system of claim 30, wherein the animate human head is that of a drug resistant epilepsy patient.

33. The system of claim 30, wherein:
the first non-invasive technique is, or includes, electroencephalography (“EEG”); and
the second non-invasive technique is, or includes, magnetoencephalography (“MEG”).

34. The system of claim 30, wherein the one or more electromagnetic properties are detected via both the first and second non-invasive techniques simultaneously.

35. The system of claim 30, wherein the instructions are executed by the one or more processors so that the following step is also executed:
deriving a source localization for a first one of the plurality of dipoles based on the one or more electromagnetic properties detected from the simulated human head via the first non-invasive technique and/or the second non-invasive technique.

36. The system of claim 35, wherein the instructions are executed by the one or more processors so that the following step is also executed:
assessing an accuracy of the first non-invasive technique and/or the second non-invasive technique, via which the one or more electromagnetic properties are detected from the simulated human head;
wherein assessing the accuracy of the first non-invasive technique and/or the second non-invasive technique comprises:
comparing the derived source localization for the first one of the plurality of dipoles with a physical location of the first one of the plurality of dipoles within the simulated human head.

37. The system of claim 36, wherein the instructions are executed by the one or more processors so that the following step is also executed:
determining, based on medical imaging, the physical location of the first one of the plurality of dipoles within the simulated human head.

38. The system of claim 30, further comprising:
the simulated human brain; and
first and second ones of the plurality of dipoles embedded in the simulated human brain;
wherein:
the first one of the plurality of dipoles is oriented tangentially within the simulated human brain; and
the second one of the plurality of dipoles is oriented radially within the simulated human brain.
